(19) United States
(12) Patent Application Publication
(10) Pub. No.: US 2025/0062021 A1
(43) Pub. Date: Feb. 20, 2025

(54) PREDICTIVE SCREENING FOR TREATMENT OF EXPIRATORY FLOW LIMITATION

(71) Applicant: KONINKLIJKE PHILIPS N.V., EINDHOVEN (NL)

(72) Inventors: Marine Flechet, Eindhoven (NL); Jan Johannes Gerardus de Vries, Eindhoven (NL); Michael Polkey, London (GB)

(21) Appl. No.: 18/678,160

(22) Filed: May 30, 2024

Related U.S. Application Data
(60) Provisional application No. 63/532,959, filed on Aug. 16, 2023.

(51) Int. Cl. G16H 50/20; A61M 16/00; G16H 10/60; G16H 40/63; G16H 50/70

(52) U.S. Cl. CPC G16H 50/20; A61M 16/0066; A61M 16/026; G16H 10/60; G16H 40/63; G16H 50/70; A61M 2205/502; A61M 2230/46

(57) ABSTRACT

An embodiment includes use of a predictive model to ascertain a likelihood of a patient having expiratory flow limitation (EFL) to adjust the application of ventilator-based therapy. An embodiment may operate one or more predictive model on patient data alone to obtain a prediction of EFL for the patient, avoiding a need to perform invasive or ventilator based EFL detection, e.g., via a forced oscillation technique (FOT). An embodiment may be used in a system or method that adjusts ventilator settings for respiratory therapy, for example to abolish detected EFL in a patient having a positive classification for EFL.

### CROSS-REFERENCE TO RELATED APPLICATIONS

This patent application claims the priority benefit under 35 U.S.C. § 119(e) of U.S. Provisional Application No. 63/532,959, filed on Aug. 16, 2023, the contents of which are herein incorporated by reference.

### BACKGROUND

### 1. Field

The disclosed subject matter generally pertains to treatment of expiratory flow limitation (EFL). Certain disclosed subject matter relates to use of a data-driven screening that identifies appropriate patients and respiratory therapy for EFL treatment.

### 2. Description of the Related Art

Exploratory Flow Limitation (EFL) is a condition that affects the ability to exhale properly, as the patient airflow is limited during exhalation. EFL usually happens in chronic obstructive pulmonary disease (COPD), but not all patients with COPD have EFL. Lungs and airways have mechanical properties that are changed due to COPD. In patients with COPD, the elastic recoil is different, in different parts of the system, and during exhalation the forces are different, and there is airway collapse. The air can go out of the patient's airway, but with more effort.

### SUMMARY

Identification and measurement of expiratory flow limitation (EFL) is not easy as it requires the use of invasive techniques or non-invasive techniques reliant on respiratory equipment, such as a forced oscillation technique (FOT) implemented in a ventilator. Once detected, EFL may be addressed, for example by adjusting the pressure setting(s) of a ventilator to manage or abolish EFL in the patient, such as increasing external positive end-expiratory pressure and titrating down to an optimal pressure value. However, application of non-invasive EFL detection to all patients is not optimal and there is a need to identify those patients for which non-invasive EFL detection is appropriate.

Accordingly, an embodiment includes use of a predictive model to ascertain a likelihood of a patient having EFL to adjust the application of ventilator-based testing and therapy. An embodiment may operate one or more predictive models on patient data alone to obtain a prediction of EFL for the patient, avoiding a need to perform invasive or ventilator based EFL detection, e.g., via a FOT. An embodiment may be used in a system or method that adjusts ventilator settings for respiratory therapy, for example to abolish detected EFL in a patient having a positive classification for EFL.

In summary, an embodiment provides a method, comprising: obtaining, using a set of one or more processors, a classification result predicting expiratory flow limitation (EFL) for one or more patients based on data associated with the one or more patients; and thereafter applying, using a ventilator, a forced oscillation technique to a set of the one or more patients having a classification result predicting EFL.

In an embodiment, a method includes determining that respective ones of the set of the one or more patients have EFL based on the results of the forced oscillation technique.

In an embodiment, the classification result is formed using a demographic feature and a spirometry test result feature. In one example, the classification result is formed using two or more of weight data, forced vital capacity (FVC) data, and data indicative of hyperinflation.

In an embodiment, the classification result is formed using a patient reported feature and a demographic feature. In one example, the classification result is formed using patient-reported data comprising one or more of: a confidence about leaving home, and an indication of being limited in activities; and one or more of: weight data, gender data, age data, and height data.

In an embodiment, a method includes classifying the one or more patients based on employing a machine learning model to analyze the data. In an embodiment, the classifying comprises employing a machine learning model to predict EFL for the one or more patients based on the data. In an embodiment, the machine learning model classifies the patients using a value for a difference in inspiratory and expiratory resistances for the one or more patients. In an embodiment, the value is related to one or more thresholds.

In an embodiment, the machine learning model is one of a patient-facing model and a clinician facing model that utilize different features to predict EFL.

In an embodiment, the method includes training a machine learning model to produce the classification result. In an embodiment, the training comprises providing a set of features for the machine learning model. In an embodiment, the set of features comprise a demographic feature and a feature derived from a spirometry test result. In an embodiment, the set of features comprise a patient-reported feature and a demographic feature.

In an embodiment, the forced oscillation technique comprises delivering a predetermined number of airflows to the one or more patients using the ventilator. In an embodiment, the method includes adjusting one or more settings of the ventilator after determining that the one or more patients have EFL.

An embodiment provides a device capable of performing one or more of the methods described herein. An embodiment provides a device, including: a display device; a set of one or more processors; and a non-transitory computer readable storage medium comprising code executable by the set of one or more processors. The code comprises: code that obtains a classification result predicting expiratory flow limitation (EFL) for one or more patients based on data associated with the one or more patients; and code that thereafter indicates, on the display device, a forced oscillation technique for a set of the one or more patients having a classification result predicting EFL.

An embodiment provides a computer program product capable of performing one or more of the methods described herein. An embodiment provides a computer program product, comprising: a non-transitory computer readable storage medium comprising code executable by the set of one or more processors. The code comprises: code that obtains a classification result predicting expiratory flow limitation (EFL) for one or more patients based on data associated with the one or more patients; and code that thereafter indicates a forced oscillation technique for a set of the one or more patients having a classification result predicting EFL.

The foregoing is a summary and thus may contain simplifications, generalizations, and omissions of detail; consequently, those skilled in the art will appreciate that the summary is illustrative only and is not intended to be in any way limiting.

These and other features and characteristics of the example embodiments, as well as the methods of operation and functions of the related elements of structure and the combination thereof, will become more apparent upon consideration of the following description and the appended claims with reference to the accompanying drawings, all of which form a part of this specification. It is to be expressly understood, however, that the drawings are for the purpose of illustration and description only and are not intended as a definition of the limits of a claimed invention.

### BRIEF DESCRIPTION OF THE DRAWINGS

FIG. 1 illustrates an example method according to an embodiment.

FIG. 2(A-B) illustrates example feature sets and importance measures of a model according to an embodiment.

FIG. 3(A-B) illustrates example feature sets and importance measures of a model according to an embodiment.

FIG. 4(A-B) illustrates example feature sets and importance measures of a model according to an embodiment.

FIG. 5(A-B) illustrates example feature sets and importance measures of a model according to an embodiment.

FIG. 6 illustrates a diagram of example system components according to an embodiment.

### DETAILED DESCRIPTION OF EXEMPLARY EMBODIMENTS

The described features, structures, or characteristics of the example embodiments may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, etc. In other instances, well known structures, materials, or operations are not shown or described in detail to avoid obfuscation.

The use of non-invasive techniques to detect expiratory flow limitation (EFL), such as use of a ventilator to perform a forced oscillation technique (FOT), requires trained clinical staff to be present with the chronic obstructive pulmonary disease (COPD) patient(s) (hereinafter “patient(s)”) using the ventilator. Next to the time spent by the clinicians, detection of EFL via a ventilator FOT also requires respiratory equipment such as a ventilator capable of detecting EFL to be available, a mask per patient, and the time, cost and effort spent by the patient to go to the hospital for the evaluation.

Embodiments overcome a problem in correctly applying a respiratory treatment, including deciding which patient should be screened for EFL using a ventilator based or invasive technique. One approach could be to screen every patient, but that leads to over application of invasive tests or of ventilator-based, non-invasive tests, each having associated and unnecessarily high costs, time spent by clinicians, and effort from the patients.

An embodiment therefore identifies patients that likely have EFL prior to selecting patients for ventilator based EFL detection techniques, as such patients will benefit from the use of the ventilators. It is noted that the term “ventilator” is defined herein to include mechanical ventilators providing invasive or non-invasive pressurized flow of breathable gas, and any other device that provides a pressurized flow of breathable gas, for example continuous positive airway pressure (CPAP) devices, bilevel positive airway pressure (BiPAP) devices, and other positive airway pressure devices. Moreover, an embodiment permits avoiding unnecessary use of ventilators and applying an unnecessary FOT to patients unlikely to have EFT. An embodiment provides predictive models that operate on patient data and allow a clinician to determine if a ventilator-based therapy such as FOT detection should be applied to the patient.

An embodiment provides for use of predictive models that predict the presence of EFL given patient data that is easily acquired. Two different predictive models are described herein by way of example. In a first example, which is referred to herein as a “clinician-facing” predictive model, the predictive model is a machine learning model that uses both demographic data and clinical data (such as data from a spirometry test) that would be available in an electronic medical record (EMR), for example after a patient performed a pulmonary function test (as is required for COPD diagnosis). The clinician facing predictive model applies mostly to a usage setting where a patient, for example during a yearly check-up, is considered for EFL testing because, for example, the current ventilation therapy does not lead to optimal results, or the patient is discovered to be hypercapnic and ventilation therapy should be initiated. A second example predictive model, which is referred to herein as a “patient-facing” predictive model, uses data that only requires patient self-reported data, such as demographics and questionnaire data, for example relating to confidence in mobility. Whereas the patient-facing predictive model might perform with slightly less accuracy and/or precision than the clinician-facing predictive model, the patient-facing predictive model might be very useful in a setting where a patient is newly considered for ventilator therapy or where the last clinician visit was long ago, or the patient is in a rural area without nearby hospitals.

The description now turns to the figures. The illustrated example embodiments will be best understood by reference to the figures. The following description is intended only by way of example, and simply illustrates certain example embodiments.

As will be appreciated by those having ordinary skill in the art, various methods outlined herein may be implemented at least in part using a computer system providing an EFL detection and compensation program that uses as input data obtained from a user, such as direct user input in response to a questionnaire, EMR data obtained from an EMR system storing spirometry tests results, demographic information, etc., or a combination of the foregoing. An embodiment therefore removes the need for invasive or ventilator-based EFL detection in certain patients and selects the most appropriate patients for ventilator-based therapies, such as for detecting EFL using a FOT and abolishment of EFL.

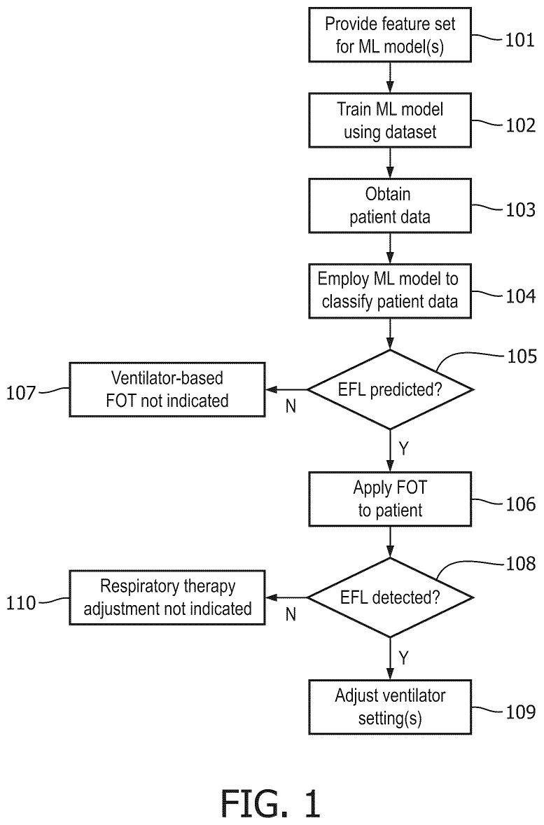

Referring to FIG. 1, an example method is illustrated. As shown, an embodiment provides for use of a trained model to evaluate patient data alone for classifying a patient as likely to have EFL. An embodiment can, with high precision, indicate which patients likely have EFL given readily available data and filter which patients are suitable for ventilator based EFL detection, applying a FOT.

As shown in FIG. 1, an embodiment may provide set of features at 101 to configure a machine learning model, for use in training as indicated at 102, or for use in a trained model at 104, used in classification tasks evaluating patient data obtained for evaluation, as indicated at 103. That is, an embodiment may include training and validating a machine learning model or may use a previously trained and validated machine learning model with a given feature set.

A feature set (including corresponding weights or coefficients) that focus a machine learning model may be provided as indicated at 101. Example data features are described further herein and may include manually curated questionnaire responses, demographic data, spirometry test results, or a combination thereof. In one example, a machine learning model may be trained to utilize a mixture of patient-supplied data (such as questionnaire responses related to confidence in mobility) and demographic data, for example reported by the patient via a graphical user interface (GUI) of a medical application, website, etc., or accessed via an electronic medical record (EMR). It is noted that all the data may be stored in an EMR if appropriate data collection techniques are employed to form such an EMR. In another example, a model may be trained to utilize a mixture of demographics and respiratory health related data, such as results of a spirometry test, as for example stored in an EMR following patient diagnosis with COPD.

A set of data features that are important for the model may be identified after some training, for example using a backwards elimination process to obtain a minimum set of features useful in the model to make the classification of interest, such as classifying a patient as likely to have EFL. In one example, a feature set is provided at 101 at least in part by manually selection, such as based on clinical expertise, and thereafter refined during training at 102 to produce a feature set used in a trained model of sufficient accuracy and precision, validated for performance against new data.

In one example, the machine learning model may predict a class indicative of EFL using training data labelled with a value representing EFL, for example a difference in inspiratory resistance and expiratory resistance (deltaXrs), which is compared to one or more thresholds to indicate EFL. In one example, the one or more thresholds include a deltaXrs value of 2.8 or 3.8, which can be used in connection with different machine learning models, as further described herein.

Once a trained machine learning model is formed, patient data obtained at 103, for example via a questionnaire, an EMR, or a combination thereof, is utilized to thereafter classify the patient data as likely to have EFL, as indicated at 104. In one example, the trained machine learning model may analyze the patient data, focused on features as configured at 101, 102, to provide an output, such as a value or classification result indicative of EFL at 105.

For patients exhibiting EFL, a ventilator-based therapy may be applied, such as a FOT, as indicated at 106. Otherwise, the patient may not be suitable for further evaluation or application of ventilator-based therapies tailored to detect or abolish EFL, as indicated at 107. For those patients predicted as having EFL at 105, subsequent application of a FOT at 106 may be used to confirm that EFL is present, at 108, and used for adjustment of ventilator setting(s), as indicated at 109. For example, confirmation of EFL via application of a FOT may result in changing ventilator settings to abolish or reduce EFL at 109, for example by applying noninvasive external positive end-expiratory pressure (EPAP) and/or an auto-titration procedure for EPAP to abolish tidal-breathing EFL using a non-invasive ventilator. Otherwise, a patient that does not exhibit EFL after ventilator-based testing at 106 may be maintained at a current therapy regimen, as indicated at 110.

Referring back to 101 and 102 of FIG. 1, in an embodiment predictive model(s) may be developed using patient data, such as from existing studies. The patient data used for training and testing includes complete data (feature sets) for a given number of patients, such as on the order of 100 patients, for the features considered for the respective predictive model, for example the clinician-facing predictive model and the patient-facing predictive model. The patient data includes an outcome, for example a label indicative of EFL, such as a result of testing for EFL using a FOT applied by a ventilator such as the BiPAP A40-EFL ventilator of Philips, measuring deltaXrs.

In the case of limited size of dataset(s), optionally model features of interest may be provided at 101, for example based expert selection of feature(s), weighting thereof, etc. Alternatively, or additionally, a larger dataset may be utilized, for example in combination with an automated search on many possible features, feature combinations, weightings, etc., to determine from the dataset a predictive model configuration. However, a suitable predictive model may be formed given a relatively small size of the dataset with some provision of manual model configuration based on feature selection, as described herein, which yields reliable and reproducible results for the predictive model.

As may be appreciated, a variety of machine learning models may be used to find and use important features, including how to combine those features in a set for a prediction of EFL. Machine learning as used herein includes methods from statistics such as, but not limited to, logistic regression as well as those originating from computing science such as, but not limited to, random forest. By way of example, techniques such as logistic regression and random forests may be utilized to identify model features of importance. Both techniques provide information on the important features used by the models to formulate predictions of EFL. One may also perform backwards feature selection to provide a minimal set of features required to reach sufficient performance, as described herein.

Predictive models, including both the patient-facing and clinician-facing predictive models, may be trained to predict an outcome associated with EFL, such as a deltaXrs value. Use of a deltaXrs value allows confirming if the predicted value is below, equal to, or above a clinically relevant threshold, for example 2.8 or 3.8. It is noted that other threshold(s) or data values may be utilized by a prediction model for predicting whether a patient is likely to have EFL. Further, a predictive model's output need not be a value for comparison with a threshold, i.e., the model may directly apply a class label to the patient data in question after being trained using data labeled with a specific value such as deltaXrs. Alternatively, or additionally, a numeric output may be supplied, e.g., a deltaXrs predicted value, which may be used with a threshold or scale to produce a prediction result or indication.

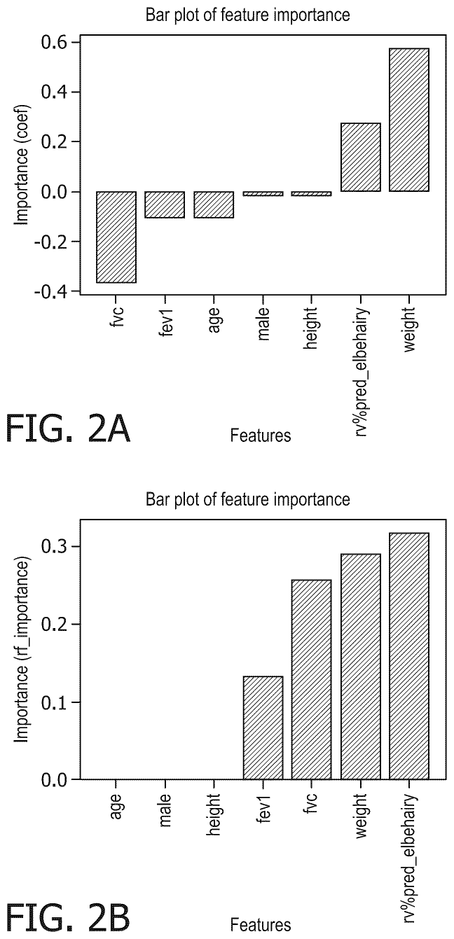

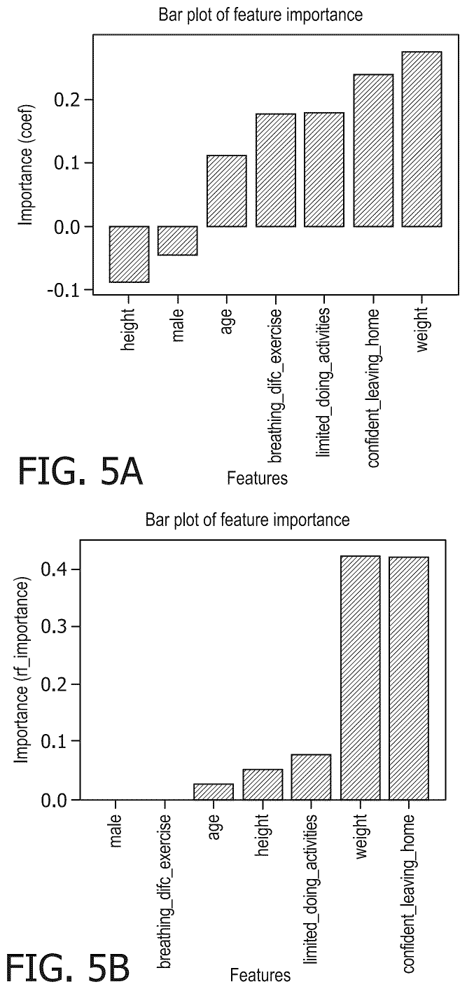

In FIG. 2(A-B) through FIG. 5(A-B), example clinician-facing and patient-facing predictive model features and their respective importance measures are illustrated. These example models were formed by a training method applied to clinical dataset, as described herein. In addition to the features illustrated in the figures, other combinations of the input features yielded worse performance, and removing further features from the resulting model feature set after backwards feature selection reduced performance significantly, when evaluated for each of the clinician-facing and patient-facing predictive models against a small clinical dataset.

Referring to FIG. 2(A-B), the clinician-facing predictive model used a threshold of deltaXrs greater than 3.8 as a decision point for labeling a patient as likely to have EFL. The example clinician-facing predictive model was provided using the following input features: age, gender, height, weight, and spirometry results. The spirometry results included forced expiratory volume, FEV1 (the amount of air that a person can force out of the lungs in 1 second), forced vital capacity, FVC (the amount of air that can be forcibly exhaled from the lungs after taking the deepest breath possible), and hyperinflation, rv % pred_elbehairy (for example, calculated as residual volume predicted by: RV %=3.58 (FVC %)−164 (FEV1/FVC)−81 (SQRT-FVC %)−0.83 (age)−10.7 (gender)+732, where: male=1, female=0).

The clinician-facing predictive model using a threshold of deltaXrs greater than 3.8 yielded performance, expressed as Area Under the Curve of the ROC curve (AUC), of 0.738 for logistic regression, and 0.678 for random forest. FIG. 2A and FIG. 2B illustrate feature importance measures identified for logistic regression and random forest, respectively. After backwards feature selection, the performances were 0.763 for logistic regression and 0.706 for random forest, where weight, FVC and hyperinflation were the three most important features for both logistic regression and random forest. By way of example, an example list of coefficients used in the clinician-facing logistic regression model is as follows:

| | | | | | | rv % |
|---|---|---|---|---|---|---|
| age | male | height | weight | fev1 | fvc | pred_elbehairy |
| −0.0082 | −0.0504 | −0.0736 | 0.0195 | −0.0150 | −0.2487 | 0.0177 |

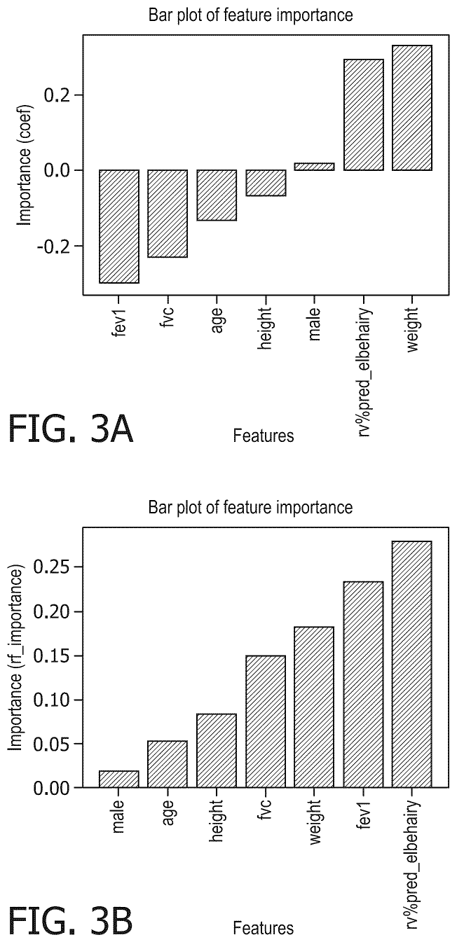

When the threshold for deltaXrs is set at 2.8 for the clinician-facing predictive model, the AUC lowers to 0.705 and 0.699 for logistic regression and random forest, respectively. The feature importance measures for the clinician facing predictive model using the lower threshold of deltaXrs (greater than or equal to 2.8) are illustrated in FIGS. 3A and 3B for logistic regression and random forest, respectively, where FIG. 3B indicates that the same four most important features are found, like use of the threshold deltaXrs greater than 3.8.

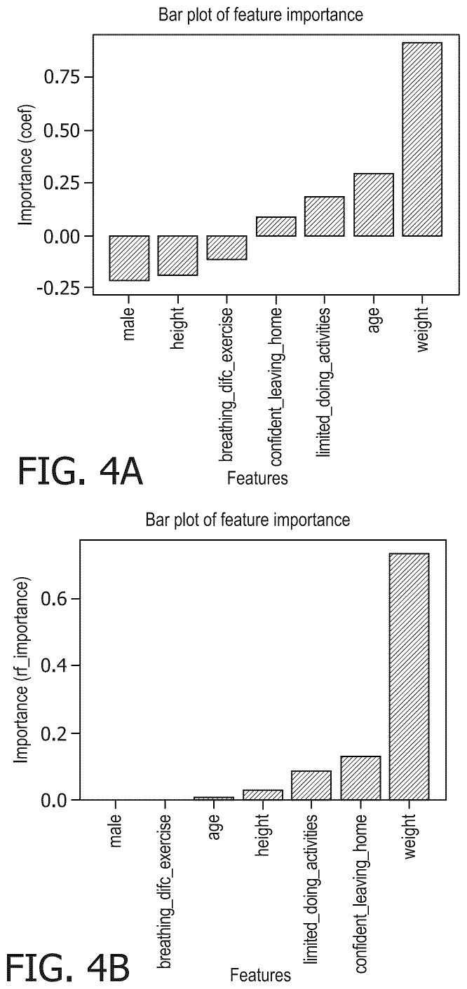

An example patient-facing predictive model included use of the following input features: age, gender, height, weight, and patient-reported disability (for example, responses to questions related to breathing difficulty when exercising, breathlessness when hill climbing, feeling limited in doing activities, and confident in leaving home). The example patient-facing predictive model yielded performance, expressed as Area Under the Curve of the ROC curve (AUC), of 0.672 for logistic regression, and 0.697 for random forest. The feature importance measures are identified for logistic regression and random forest in FIG. 4A and FIG. 4B, respectively.

After backwards feature selection, the performance was 0.715 for logistic regression and 0.717 for random forest. Weight, gender, and age were the three most important features for logistic regression. Weight, confident_leaving_home, limited_doing_activities, and height were the most important features for random forest. Backwards feature selection suggested removal of one or more of the questionnaire-based features, indicating that other optimal questionnaire-based features may be identified for future models using the techniques described herein.

An example list of coefficients used in the patient-facing logistic regression model is as follows:

| | | | | Breathing | Limited | Confident |
|---|---|---|---|---|---|---|
| | | | | difficulty | doing | leaving |
| age | male | height | Weight | exercise | activities | home |
| 0.0032 | −0.0003 | −0.0025 | 0.0145 | 0.0003 | 0.0012 | 0.0011 |

For the example patient-facing predictive model, using a deltaXrs threshold of greater than or equal to 2.8, the AUC lowers to 0.672 and 0.655 for logistic regression and random forest, respectively. The feature importance measures are illustrated in FIGS. 5A and 5B for logistic regression and random forest, respectively.

As may be appreciated, use of the example predictive models or other predictive models formed via a similar process may yield features of patient data, for example EMR data, patient response to questions, etc., that may be used to form rules-based systems for determining eligibility for ventilator-based EFL detection and indicate settings for application of such tests, customized to the patient's needs. In an embodiment, a rules-based system may be utilized alone or in combination with the use of machine learning predictive models.

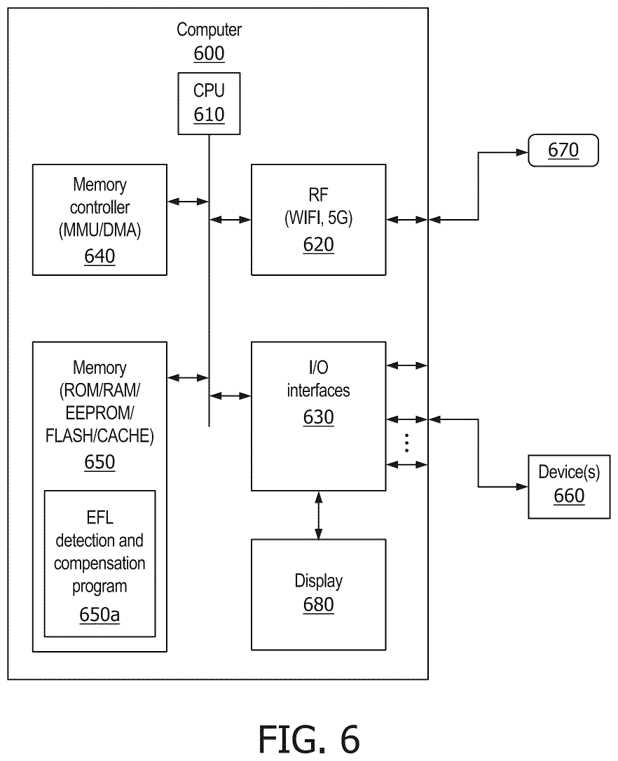

Referring to FIG. 6, it will be readily understood that certain embodiments can be implemented using any of a wide variety of devices or combinations of devices and components. In FIG. 6 an example of a computer 600 and its components are illustrated, which may be used in a device for implementing the functions or acts described herein, e.g., forming and training a predictive model, classifying of patient data using a trained predictive model, forming, or providing ventilator-based tests, applying ventilator setting adjustments, etc. Also, circuitry other than that illustrated in FIG. 6 may be utilized in one or more embodiments. The example of FIG. 6 includes certain functional blocks, as illustrated, which may be integrated onto a single semiconductor chip to meet specific application requirements.

One or more processing units are provided, which may include a central processing unit (CPU) 610, one or more graphics processing units (GPUs), and/or micro-processing units (MPUs), which include an arithmetic logic unit (ALU) that performs arithmetic and logic operations, instruction decoder that decodes instructions and provides information to a timing and control unit, as well as registers for temporary data storage. CPU 610 may comprise a single integrated circuit comprising several units, the design and arrangement of which vary according to the architecture chosen.

Computer 600 also includes a memory controller 640, e.g., comprising a direct memory access (DMA) controller to transfer data between memory 650 and hardware peripherals. Memory controller 640 includes a memory management unit (MMU) that functions to handle cache control, memory protection, and virtual memory. Computer 400 may include controllers for communication using various communication protocols (e.g., I²C, USB, etc.).

Memory 650 may include a variety of memory types, volatile and nonvolatile, e.g., read only memory (ROM), random access memory (RAM), electrically erasable programmable read only memory (EEPROM), Flash memory, and cache memory. Memory 650 may include embedded programs, code, and downloaded software, e.g., EFL detection and compensation program 650a that provides coded methods such as illustrated and described in connection with FIG. 1 (or part thereof). By way of example, and not limitation, memory 650 may also include an operating system, application programs, other program modules, code, and program data, which may be downloaded, updated, or modified via remote devices.

A system bus permits communication between various components of the computer 600. I/O interfaces 630 and radio frequency (RF) devices 620, e.g., WIFI and telecommunication radios, may be included to permit computer 600 to send data to and receive data from remote devices using wireless mechanisms, noting that data exchange interfaces for wired data exchange may be utilized. Computer 600 may operate in a networked or distributed environment using logical connections to one or more other remote computers or devices 670, such as a ventilator. The logical connections may include a network, such local area network (LAN) or a wide area network (WAN) but may also include other networks/buses. For example, computer 600 may communicate data with and between device(s) 660, for example user device(s) that provide responses to questionnaires, database(s) storing EMRs, servers storing trained machine learning prediction models that respond to application programming interface (API) calls with a classification result, etc.

Computer 600 may therefore execute program instructions or code configured to obtain, store, and analyze patient medical data and perform other functionality of the embodiments, such as described in connection with FIG. 1. A user can interface with (for example, enter commands and information) the computer 600 through input devices, which may be connected to I/O interfaces 630. A display 680 or other type of output device may be connected to or integrated with the computer 600, for example via an interface selected from I/O interfaces 630.

It should be noted that the various functions described herein may be implemented using instructions or code stored on a memory, e.g., memory 650, that are transmitted to and executed by a processor, e.g., CPU 610. Computer 600 includes one or more storage devices that persistently store programs and other data. A storage device, as used herein, is a non-transitory computer readable storage medium. Some examples of a non-transitory storage device or computer readable storage medium include, but are not limited to, storage integral to computer 600, such as memory 650, a hard disk or a solid-state drive, and removable storage, such as an optical disc or a memory stick.

Program code stored in a memory or storage device may be transmitted using any appropriate transmission medium, including but not limited to wireless, wireline, optical fiber cable, RF, or any suitable combination of the foregoing.

Program code for carrying out operations according to various embodiments may be written in any combination of one or more programming languages. The program code may execute entirely on a single device, partly on a single device, as a stand-alone software package, partly on single device and partly on another device, or entirely on the other device. In an embodiment, program code may be stored in a non-transitory medium and executed by a processor to implement functions or acts specified herein. In some cases, the devices referenced herein may be connected through any type of connection or network, including a local area network (LAN) or a wide area network (WAN), or the connection may be made through other devices (for example, through the Internet using an Internet Service Provider), through wireless connections or through a hard wire connection, such as over a USB connection.

An embodiment may be implemented in a variety of devices, including user devices such as a smartphone or tablet running a mobile application. In one embodiment, referring to FIG. 1, the obtaining of patient data 103 and employing a machine learning model 104 are performed locally on a client device such as a smartphone running a mobile application. In such an embodiment, a trained model such as a patient-facing prediction model may be downloaded and run locally on the user device. For example, an application program, such as an EFL detection and compensation program 650a as described herein, may include predictive model and one or more application programming interfaces (APIs) to call a machine learning (ML) subsystem of the user device, for example calling an inferencing API of CORE ML on Apple's IPHONE. In some embodiments, the predictive model version (e.g., patient-facing or clinician-facing) may be selected (e.g., downloaded, activated for running, etc.) based on identified characteristics or contextual data. For example, a predictive model trained to predict EFL based on patient questions may be selected, for example by a user or as part of an application routine, based on knowledge of which type of device or user is requesting the predictive output, the data availability, etc. Therefore, one or more predictive models may be used in different circumstances, e.g., by patients or clinicians.

Therefore, an embodiment may include an application program configured to execute computer program instructions, for example as outlined at least in part in FIG. 1, which in combination with local user device hardware, permit access to and classification of medical data. Such an embodiment permits the user's confidential medical data, including responses to questions, to remain solely on the user's device, increasing confidence of the user in applying any analysis or machine learning techniques to the user's medical data.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” or “the” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination. The word “about” or similar relative term as applied to numbers includes ordinary (conventional) rounding of the number with a fixed base such as 5 or 10.

It is worth noting that while specific blocks are used in the figures, and a particular ordering of blocks has been illustrated, these are non-limiting examples. In certain contexts, two or more blocks may be combined, a block may be split into two or more blocks, or certain blocks may be re-ordered or re-organized or omitted as appropriate, as the explicit illustrated examples are used only for descriptive purposes and are not to be construed as limiting.

As used herein, the statement that two or more parts or components are “coupled” shall mean that the parts are joined or operate together either directly or indirectly, e.g., through one or more intermediate parts or components, so long as a link occurs. As used herein, “operatively coupled” means that two or more elements are coupled to operate together or are in communication, unidirectional or bidirectional, with one another. As used herein, the term “number” shall mean one or an integer greater than one (i.e., a plurality). As used herein a “set” shall mean one or more.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A method, comprising:
obtaining, using a set of one or more processors, a classification result predicting expiratory flow limitation (EFL) for one or more patients based on data associated with the one or more patients; and
thereafter applying, using a ventilator, a forced oscillation technique to a set of the one or more patients having a classification result predicting EFL.

2. The method of claim 1, comprising determining that respective ones of the set of the one or more patients have EFL based on the results of the forced oscillation technique.

3. The method of claim 1, wherein the classification result is formed using two or more of weight data, forced vital capacity (FVC) data, and data indicative of hyperinflation.

4. The method of claim 1, wherein the classification result is formed using:
patient-reported data comprising one or more of: a confidence about leaving home, and an indication of being limited in activities; and
one or more of: weight data, gender data, age data, and height data.

5. The method of claim 1, comprising classifying the one or more patients based on the data.

6. The method of claim 5, wherein the classifying comprises employing a machine learning model to predict a level of EFL for the one or more patients based on the data.

7. The method of claim 6, wherein the machine learning model classifies the one or more patients using a value for a difference in inspiratory and expiratory resistances for the one or more patients.

8. The method of claim 7, wherein:
the value is predictive of EFL; and
the value is related to one or more thresholds.

9. The method of claim 6, wherein the machine learning model is one of a patient-facing model and a clinician facing model.

10. The method of claim 1, comprising training a machine learning model to produce the classification result.

11. The method of claim 10, comprising providing a set of features for the machine learning model.

12. The method of claim 11, wherein the set of features comprise a demographic feature and a feature derived from a spirometry test result.

13. The method of claim 11, wherein the set of features comprise:
a patient-reported feature; and
a demographic feature.

14. The method of claim 1, wherein the forced oscillation technique comprises delivering a predetermined number of airflows to the one or more patients using the ventilator.

15. The method of claim 2, comprising adjusting one or more settings of the ventilator after determining that the one or more patients have EFL.

16. A device, comprising:
a display device;
a set of one or more processors; and
a non-transitory computer readable storage medium comprising code executable by the set of one or more processors, the code comprising:
code that obtains a classification result predicting expiratory flow limitation (EFL) for one or more patients based on data associated with the one or more patients; and
code that thereafter indicates, on the display device, a forced oscillation technique for a set of the one or more patients having a classification result predicting EFL.

17. The device of claim 16, wherein the classification result is formed using two or more of weight data, forced vital capacity (FVC) data, and data indicative of hyperinflation.

18. The device of claim 16, wherein the classification result is formed using:
patient-reported data comprising one or more of: a confidence about leaving home, and an indication of being limited in activities; and
one or more of: weight data, gender data, age data, and height data.

19. The device of claim 16, comprising code that classifies the one or more patients based on the data;
wherein the code that classifies comprises code that employs a machine learning model to predict EFL for the one or more patients.

20. A computer program product, comprising:
a non-transitory computer readable storage medium comprising code executable by the set of one or more processors, the code comprising:
code that obtains a classification result predicting expiratory flow limitation (EFL) for one or more patients based on data associated with the one or more patients; and
code that thereafter indicates a forced oscillation technique for a set of the one or more patients having a classification result predicting EFL.
